(19) United States
(12) Patent Application Publication
(10) Pub. No.: US 2008/0213821 A1
(43) Pub. Date: Sep. 4, 2008

(54) Microfluidic Cell Sorter System

(75) Inventors: Ai Qun Liu, Singapore (SG); Xiao Jun Liang, Singapore (SG); Xu Ming Zhang, Singapore (SG); Yi Sun, Singapore (SG); Peter Droge, Singapore (SG)

Correspondence Address: KNOBBE MARTENS OLSON & BEAR LLP, 2040 MAIN STREET, FOURTEENTH FLOOR, IRVINE, CA, 92614, US

(73) Assignee: NANYANG TECHNOLOGICAL UNIVERSITY, Singapore (SG)

(21) Appl. No.: 11/579,074

(22) PCT Filed: May 6, 2005
(86) PCT No.: PCT/SG05/00142

§ 371 Date: Sep. 10, 2007

Related U.S. Application Data
(60) Provisional application No. 60/568,266, filed on May 6, 2004.

(51) Int. Cl. C12M 1/34; C12M 1/36; C12Q 1/20; H05H 1/00

(52) U.S. Cl. 435/39; 435/286.5; 435/34; 435/288.7; 264/219; 264/483

(57) ABSTRACT

A microfluidic system for separating, purifying and counting cell sub-populations, utilising steering of liquid flows in microfluidic channels in a cell focusing region (first dotted circle area); having the integration of the optical detection mechanism and a microchannel structure made from moulding. A master is photolithographically patterned on a soft PDMS silicon or polymer material. After being moulded and peeled off the master, the micro-channel structure is sealed on a hard substrate with openings punched through for wells (14, 12, 36, 38, 40). An optical detection region (20) discriminates different types of cells that have been formed into a single flow (30). Electromagnetic fields are used to steer (32) the flows of cells according to the signals from the optical detection region into branch channels leading to the punched wells for separate collection. The system can have parallel systems that increase throughput or cascade systems to provide several analysis steps. The optical system an micro-lens (24) for the system can be imbedded in the moulding material during formation of the mould.

### RELATED APPLICATIONS

This International Phase PCT application claims priority from the U.S. Provisional Application 60/568,266, filed on May 6, 2004 priority of which is claimed and which is hereby incorporated by reference.

### TECHNICAL FIELD

This invention relates to cell sorting systems used in medical diagnoses and biological studies. It also relates to the method of making such a sorter system and the method of using the system for sorting.

### BACKGROUND ART

This invention relates to cell sorter systems using polymer- or silicon-based microfluidic channels and their applications in medical and biological diagnoses.

In biomedical studies, preparation of samples prior to a detection step tends to be relatively complicated. Multiple specimen types and different types of target cells must be processed. The manipulation and separation of particles, especially living cells, is a basic step for many biological and medical applications, including isolation and detection of sparse cancer cells, concentration of cells from dilute suspensions, separation of cells according to specific properties, and trapping and positioning of individual cells for characterization. Among various technologies for these purposes, microfluidic systems based on microelectromechanical systems (MEMS) technologies have attracted scientific and industrial attention since their introduction in the early 1990s. Many works have been focused on using electrokinetic forces to separate analytes such as peptides, DNA fragments and cells through capillary electrophoresis in a single chip. It understood that microfabricated devices provide one or more advantages of small size, easy operation and low cost.

Conventional flow cytometry and fluorescence-activated cell sorters (FACS) are widely used in clinical medicine, basic biological and material sciences. FACS provides impressively efficient sorting. However, a FACS is expensive, and requires relatively large sample volumes. In addition, it is difficult to sterilize and is mechanically complicated, and can only be operated and maintained by trained personnel. Therefore, inexpensive devices that can rapidly and efficiently sort live cells, particles and even single molecules would greatly facilitate biological science research and medical diagnosis. Recently, development of miniaturized cell interrogation and sorting tools are of great interest for portable diagnostic instruments. Kruger et al. demonstrated a miniaturized flow cytometer which can perform the key functions of detection, enumeration and sorting of fluorescent species. A. Y. Fu et al. described their efforts in developing a microfabricated elastometric cell sorter. Using electrokinetic flow, they demonstrated sample dispensing, interrogation, automation, sorting and recovery. Dittrich et al. reported an integrated micro total analysis system based on a microfluidic on-chip device for reaction, high-sensitivity detection and sorting of fluorescent cells and particles. L-M Fu et al. also demonstrated a microflow cytometer using electrokinetic forces for flow switching with buried optical fibers for on-line detection. Various types of biochemical reactions have also been successfully carried out within microcapillary systems, including enzymatic and immunoassays and polymerase chain reactions, with low sample consumption, short reaction times due to efficient heat transfer, and with low production and operating costs of respective microchips.

Most microfabricated cell sorters are based on conventional semiconductor materials and techniques originated from integrated circuits. Microfluidic devices using those materials and techniques are not only expensive, but also have many limitations on fabrication, packaging and testing. For example, most of biological experiments require the material to be hydrophilic so that the microflow can be easily manipulated. Unfortunately, most semiconductor materials are hydrophobic. Recently, several alternative technologies using organic polymers have been proposed, for example, SU-8 and polydimethylsiloxane (PDMS). Unlike traditional semiconductor material such as silicon and glass, PDMS is a low-cost polymer. It is soft, elastic and easy to process. PDMS micromolding techniques have been used for fabrication of MEMS and microfluidic systems. This process is simple and rapid in comparison to traditional etching and bonding methods. In addition, PDMS has the advantages of easy bonding, good optical properties (transparent from 230 nm to 700 nm) and permeability to gases. Therefore, PDMS is particularly suitable for fabricating various microfluidic devices.

Several types of physical forces have been employed for particle manipulation, including those of mechanical, hydrodynamic, ultrasonic, optical and electromagnetic origins. However, electroosmosis force (EOF) is of most interest. Electroosmosis is the pumping effect generated in a fluid within a channel under the application of an electrical field. Above pH 2, a negative surface charge characterized by the zeta potential exists at the plane of shear between the stationary and mobile layers of the electric double layer (EDL). The zeta potential is typically on the order of −20 mV to −150 mV. The surface charge comes either from the wall property or the absorption of charged species in the fluid. In the presence of an electrolyte solution, the surface charge induces the formation of a double layer on the wall by attracting oppositely charged ions from the solution. This layer has a typical thickness on the order of nanometers. An external electrical field forces the double layer to move. Due to the viscous force of the fluid, the whole fluid in the channel moves until the velocity gradient approaches zero across the microchannel.

This effect results in a flat velocity profile. After applying the electrical field, the momentum transfer process is on the time scale between 100 μs and 1 ms. The electroosmotic flow velocity u_{eof} is obtained by

u_{eof}=μₑₒEₑ₁ (1)

where μₑₒ is the electoosmotic mobility of the fluid and Eₑ₁ is the electric field strength. μₑₒ is a function of the dielectric constant of the solvent ∈, its viscosity η, and the zeta potential ξ as given by

$${μ}_{eo} = \frac{ɛζ}{η} \qquad ( 2 )$$

Due to its nature, the electroosmosis effect is good at pumping fluid into small channels without a high external pressure. In microanalysis systems, electroosmosis is used for delivering a buffer solution in combination with the electrophoretic effect for separating molecules. Mass transport in the microfluidic network is also possible using pressure-driven flows. In theory, pressure-driven flows do not downscale well since smaller channel dimensions require a higher pressure drop in order to maintain the flow velocity. The major difference between electroosmotic and pressure-driven flow lies in the velocity profile in the channel. In case of pressure-driven flows, the flow velocity is zero at the channel wall, and gradually increases towards the center of the channel, according to a parabolic profile. Therefore, a section of fluid introduced into a channel will be distorted upon transport as the fluid in the central part of the channel moves faster than that close to the walls. On the contrary, electroosmotic transport is characterized by a uniform distribution of flow velocities over the channel, except very close to the channel walls.

Most commercially available similar microfluidic cell sorter systems in the market apply pressure-driven methods to pump fluid in their devices. But for the present cell sorting system, electroosmotic flow is used as the mechanism to transport sample solution through the channel systems. This provides several advantages. First, separations are more efficient using electroosmosis than by using pressure-driven flows, due to lower dispersion by the flow profile. Secondly, electroosmotic pumping is a convenient tool for tuning liquid flow in individual channels, by varying the applied voltages at each channel end. Furthermore, since the response of electroosmotic flow to the direction of the applied field is instantaneous, electrical manipulation provides much shorter switching times and thus, yields much higher sorting speeds. Last but not the least, electroosmotic manipulation on-chip eliminates the need for an external syringe pump, hence, is suitable for miniaturization as it is relatively easy to generate and structure an electrical field at microscale.

### DISCLOSURE OF THE INVENTION

One feature of this invention is the overall design and integration of a microchannel structure, an optical detection method and a switching mechanism. For example, compared with a recent work by L-M Fu et al. (16), the present invention employs angled channels for pulsated injection switching, a microlens set for optical coupling, fluorescence emission for cell differentiation, molding of polymer for fabrication, and on-chip light source for illumination. In contrast, L-M Fu et al. (16) used assisted channels for switching, no lens system for optical coupling, light scattering for cell differentiation, wet etching of soda lime glass for fabrication, an external light source and embedded optical fiber waveguide for illumination.

Thus, the present invention solves one problem of the prior art by providing a cell sorting system which is compact, has low cost, is disposable, has no cross contamination, allows a small sample volume, high accuracy and high throughput. The present invention also allows simple and inexpensive fabrication of the system.

According to one aspect of the invention, a microfluidic cell sorter includes a hard substrate, a microchannel structure made of soft material (such as a polymer), an optical detection subsystem, and an electrical circuit. Typically, the hard substrate is a glass slide, a silicon wafer or a polymer slab. The integrated optical subsystem contains an embedded light source, a micro-lens array and a photo detector for light input and output. The light source can be either a light emitting diode (LED), a laser diode (LD), or an organic LED (OLED), etc. (LEDs are shown in the drawings of this patent as an illustrative example). Typically the electrical circuit consists of electrodes and wires made of gold, platinum or other metal. It should also include a controller for signal and data processing. The controller is a separate component for data processing. It can either be embedded on the hard substrate to form a system-on-a-chip apparatus, or be positioned external to the hard substrate. In both cases, the controller is connected to the electrodes, the light source and the photo detector, etc. through electrical wires. From the functional point of view, a cell sorter system has at least one analysis unit. An analysis unit provides the basic functions of cell separation and cell counting, and consists of a microfluidic structure for flow control and cell transportation, an optical detection region for cell discrimination, an electrical circuit to apply an electrical field to steer the flow, and a hard substrate for sealing and supporting the microfluidic structure. In certain embodiments, the cell sorter system has many analysis units arranged in cascade and parallel.

In certain embodiments, the fabrication of the cell sorter system starts with photolithographically patterning a layer of hard material (such as SU-8 photoresist (from MICROCHEM Corp and SOTEC Microsystems), quartz and silicon) having the desired thickness. After patterning, the remaining part of the hard material forms a master. Then the master is molded using a soft material (such as PDMS, polyethylene, polystyrene and other biocompatible elastomers). In certain embodiments, the optical detection subsystem is embedded into the soft material during the molding. In a typical process, the optical subsystem is put on the mold and carefully positioned before the molding; then the soft material is poured in to cover the mold and the optical subsystem as well; after the soft material is baked, the optical subsystem is firmly embedded inside the soft material.

After peeling off the soft material, the microchannel structure is bonded on a hard substrate for sealing the microchannels. In certain embodiments, the hard substrate is patterned with a thin layer of metal (such as gold or platinum) to provide the desired electrical connection. In a typical fabrication process, a layer of metal is deposited on the substrate, and is then patterned using a photomask (17). The electrical connection can be on the same side of the substrate with microchannel structure, or on the other side, or on both sides. As an electrical field is needed to steer the flow, there should be electrical pads directly under the wells of the microchannel structure in the bonded biochip. Therefore, some electrical pads and wires should be on the same side of the microchannel. The other pads are for connection to the voltage source (for EOF driving), current source (for LED driving), and for transmitting electrical signals such as optical detection signals from the photodetector. External metal (preferably gold or copper) wires may be bonded from these pads to the external components (voltage/current sources, photodetector, and controller etc.). Therefore, these pads can be on the opposite side of the microchannel structure, or on the same side.

In one of the analysis units, from the inlets to the outlets, the cell mixture typically needs to pass through three functional regions in series: a cell focusing region, an optical detection region and a cell switching region. After the cell mixture enters the main channel from the inlet, the cells first flow to a cell focusing region. The function of cell focusing is to line up the originally randomly-positioned cells and let them flow one by one with a certain desired spacing. In the optical detection region, the cells are examined one by one when they pass through the focused light beam. Detection of optical properties of the individual cells helps to identify the cell type, and generates a controlling/counting signal to the electrical circuit. In the cell switching region, when the cells further flow to the intersection of the branch channels and the main channels, they are steered to the targeted branch channels based on the type of the individual cells. The flow direction is controlled by the electrical field through the EOF. The direction of the electrical field is controlled by signals from the controller that change the voltage at the electrodes located at various wells.

The various embodiments may have one or more of the following advantages.

The cell sorter system can be very cheap as the structure is formed by photolithography and molding. Many cell sorter systems can be fabricated in the same process. The materials involved are mainly cheap materials such as polymers and glass. In addition, the fabrication does not need a high-performance clean room. The low cost may pave the way for the cell sorter system to be used in clinical diagnosis.

The cell sorter system is disposable and thus avoids cross contamination, which is a serious problem in the conventional re-useable cytometer.

The cell sorter system is compact compared with the commercial huge cytometer machine. A typical dimension is 2 cm×2 cm×5 mm.

A small and cheap light source such as LED, LD or OLED is used to replace the conventionally used, large and expensive laser source, which greatly improves integrity and reduces the cost of the cell sorter system.

The sorting speed is largely increased by the unique injection switching design. 45 degree switching channels operate with a switch time as low as several hundred nano seconds.

The sorting accuracy is very high as the cells are examined one by one. The accuracy can be further improved or multiple analyses can be serially performed by cascading several analysis units. In addition, the sample can be very sparse (e.g., 1 to 1000 particles per microliter) and have a very small volume (≦1 nanoliter).

The cell sorter system can be run automatically and does not need experts to operate it.

The sorting throughput is high as the flow is steered by the electroosmotic force, which responds instantaneously to the electrical field. The sorting throughput can be further improved by paralleling several analysis units.

### BRIEF DESCRIPTION OF DRAWINGS

FIG. 1 according to the present invention is a diagrammatic view of the cell sorter system;

FIG. 2 is a schematic view of a cell sorter system having cascaded levels of analysis units;

FIG. 3 is a close-up view of the cell focusing region in the cell sorter system;

FIG. 4(a) is a close-up view of the cell detection region, showing LED light and the integrated optical subsystem;

FIG. 4(b) is a diagrammatical view of an embodiment of the optical detection system, which employs microlens sets to focus the input LED light and to collect the output light;

FIG. 4(c) is a close-up view of a microlens set;

FIG. 5(a) is a diagram illustrating a simple three-well angled-channel microfluidic structure and its fluidic field pattern, which shows the flow leakage during switching;

FIG. 5(b) is a diagram illustrating a vertical-angle microchannel structure and its fluidic field pattern;

FIG. 5(c) is a graph showing the relationship between the switching time and the channel angle θ;

FIG. 5(d) is a graph showing the relationship between the flow leakage and the channel angle;

FIG. 6 show the fabrication steps of one example of the cell sorter system, in which, SU-8 is used as the mastering material, PDMS as the molding material, and a glass slide as the hard substrate.

### BEST MODES FOR CARRYING OUT THE INVENTION DETAILED DESCRIPTION

### Definitions

The terms used in this specification generally have their ordinary meanings in the art. However, to better understand the invention, it is helpful to clarify the meaning of certain terms.

The term “cell” refers to prokaryotic and eukaryotic cells, including their various components. In addition, particles other than cells, having a microscopic size from about 10 nm to about 1 mm, including but not limited to viruses, protein complexes, molecules, micro beads, particles of various composition, liposomes, and emulsions etc. can also be analyzed using the present invention.

The term “cell sorter system” refers to all the necessary parts for the cell sorting function. It consists mainly of a microchannel structure, a hard substrate (e.g. glass slide), an optical detection subsystem, and an electrical circuit.

The term “microchannel structure” refers to all the channels for containing and flowing the cells, including but not limited to, inlets, outlets, wells, focusing channels, main channels, branch channels.

The term “hard substrate” is a substrate with certain thickness and rigidity for sealing and supporting the microchannel structure, for example, a glass slide, a silicon wafer, or a polymer slab.

The term “biochip” refers to the hard substrate and all the other components integrated on the hard substrate. In certain embodiments, the cell sorter system may have two parts, a platform part and a biochip part. The platform part includes all the re-useable components (such as power supply, current source, controller and photodetector etc.) while the biochip part is disposable. In the diagnosis, the sample is first put into the biochip, then the biochip is put into the platform part. The platform part runs the biochip automatically, measures and analyzes the data and displays information. Then the biochip can be pulled out and thrown away. Before disposal, the collected cells can be retrieved for further investigation by various means, for example, using a micropipetfe to suck them out.

The term “optical detection subsystem” refers to the optical components for optical detection, including but not limited to optical fibers and waveguides for the input and the output light and, the light sources, the microlens sets for compressing the input light beam and for collecting the output light, and any desired optical filters. The optical detection system may also include other devices external to the cell sorter, such as photodetectors, spectrometers, etc. In certain embodiments, the external optical devices are positioned with respect to the biochip to maintain optimal coupling of the optical signals from the biochip to the external optical devices for characterization. For example, a collecting lens set on the biochip may direct light into a photodetector or into an optical waveguide on the platform.

The term “electrical circuit” refers to the necessary components for applying the electrical field to control the flow direction and flow rate. It includes but is not limited to the electrodes, and the wires. The circuit may also include devices external to the system such as a power supply and a controller, which are connected to the biochip through electrical wires by wire bonding or by contact pads.

The term “analysis unit” refers to a part of the cell sorting system which provides the basic function of cell sorting and counting. In certain embodiments, a plurality of analysis units arranged in cascade to improve the sorting accuracy and/or to provide serial analyses and sorting. In certain embodiments, multiple analysis units are arranged in parallel to provide high sorting throughput. Both arrangements may be combined in one sorter device. Typical throughput and accuracy for one analysis unit is 5-70 events per second and 90%-95%, respectively. By paralleling and cascading, the performance can be improved to >1000 events per second at >99% accuracy.

The term “sorting accuracy” is defined as the number of the desired cells in a targeted outlet over the total number of the cells that are steered to this outlet.

The term “sorting throughput” is defined as the number of cells that are sorted per unit of time.

Cell Sorter Architecture and Method

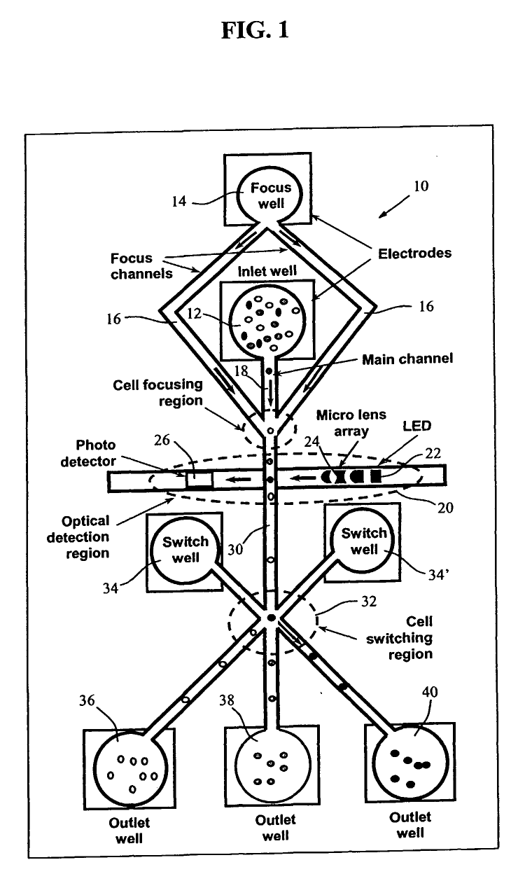

A simple cell sorter system 10 is shown in FIG. 1. This system has only a single analysis unit for sorting according to one criterion. The original sample is placed in inlet well 12. The sample includes some cells having the criterion for selection and other cells which do not.

The cell mixture is transported from the inlet well into the main channel 18 by an electroosmotic force, which is controlled by the electrodes at the inlet and outlet wells.

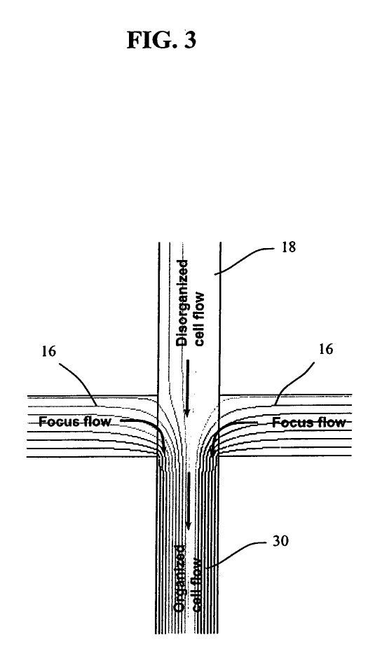

A focus well 14 includes a solution containing no cells, such as plain water or buffer. The solution travels through focus channels 16 to the cell-focusing region where these channels meet the main channel 18. The flow from the focus channels travels along the sides of the resulting main channel 30, as seen in FIG. 3. This causes the flow of solution containing the cells in the main channel to be focused to the midpoint in the stream. This also causes the cells to be separated.

When the cells pass through the integrated optical subsystem 20, they are subjected to light from a light emitting diode 22. This light passes through a microlens array 24 before shining on the cells. A photo detector 26 picks up the resulting light and produces an output, which can distinguish different cells. The photodetector may be remote from the sorter, being connected thereto by an optical waveguide, such a glass fiber or photonic crystal waveguide. In such an embodiment, the aperture face of the waveguide collects light from the cells.

The cells continue through the main channel 3 until reaching a cell switching region 32. At this point, the electrodes are controlled to change the electroosmotic force so that fluid is drawn from switch wells 34 to cause the flow to progress into one of the three branches which reach outlet wells 36, 38 and 40. For example, if a cell is supposed to go to the outlet 36, the potential at the outlet 36 should be set to 0 V while the outlets 38 and 40 should be left as an open circuit (refer to FIG. 1). At the same time, the inlet well 12 and the focus well 14 have applied thereto a potential of 100 V to generate the flow in the main channel 18 and the focus flow in the focus channel 16. The switching well 34 is subjected to a potential of 50 V to push the targeted cell to the outlet 36. The other switching well 34 is also left as an open circuit. Once the cells are recognized, the cell switching region allows different kinds of cells to be collected in different outlet wells.

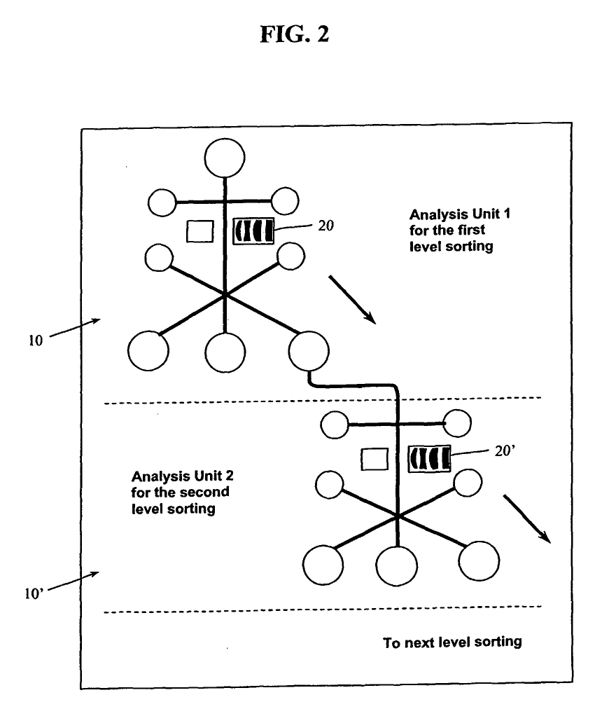

FIG. 2 shows a more complex unit where two cascaded analysis units are provided. In this system, a first analysis unit 10 is used to separate the cells into three outlet wells, in the same fashion as FIG. 1. However, the cells from one of the wells are then used as an input to the second analysis unit 10′ for a sorting using a different criterion. These cells are then collected in the outlet wells at the bottom of the second unit. If desired, further analysis criteria can be used in different levels. As an example, the first analysis unit can be used to separate white cells from other cells, such as red cells and platelets, etc. In the second level sorting, the second analysis unit is used to separate five different types of white cells. It would also be possible for the second level to be used for additional sorting accuracy. Thus, if the first sorting is only 90% correct, a second sort may improve the accuracy to 99%.

It would also be possible to utilize similar level analysis units in parallel in order to increase the throughput of the cell sorting.

### Cell Focusing Using Electrokinetic Flow Control

Referring to FIG. 3, the initially disorganized cells are lined up by cell focusing. In the main channel 18 from the inlet well, the cells are randomly positioned. At the intersection of the cell focusing region, the cell flow and the focus flows meet and form main channel 30. Since the focus flows occupy a certain amount of space in the channel, the space for the cell flow is narrowed. By controlling the flow rate for the focus flows, the space for the cell flow can be narrowed to about the size of a single cell. The result is that the cells can only pass the intersection region one at a time. Thus, the cells are focused at the center of the stream and a spacing is provided between the cells. This guarantees a single cell flow through the detection region so that cells are accurately counted and more sensitive measurements can be obtained. Thus, by electrokinetically focusing the flow within the transporting channels, there is no need for extremely narrow channels which are difficult to handle and suffer from frequent clogging.

In order for the cell flow to be focused into the center of the stream, the focus flow should be well balanced. By using a single focus well, the hydrostatic pressure will remain the same in the two different flow channels, thus avoiding the possible influence of a hydrostatic pressure difference.

### Cell Discrimination Using Optical Detection

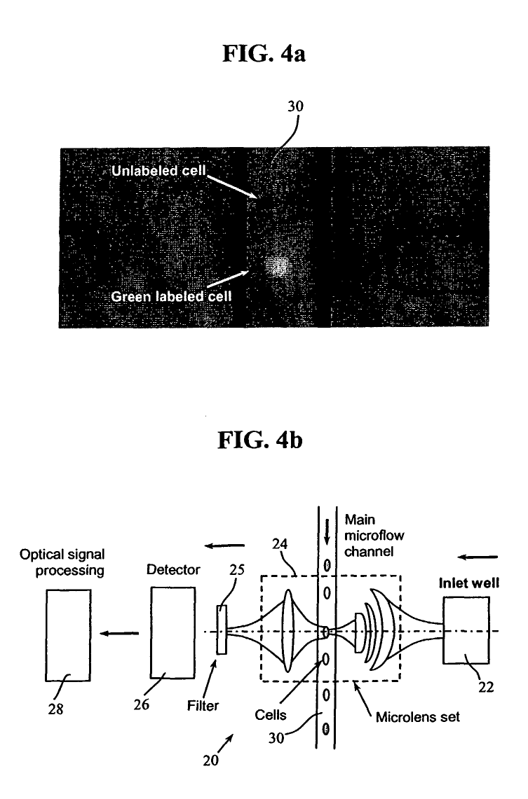

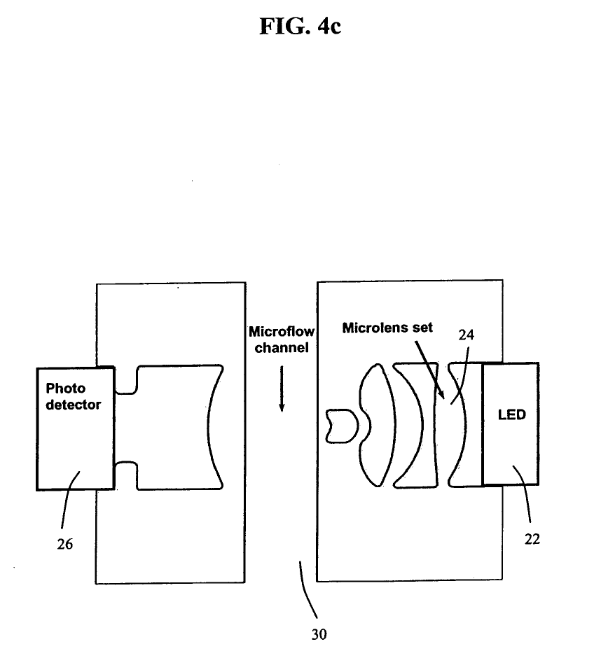

FIGS. 4a, b and c show the arrangement of the optical system used to detect the cells. As seen in FIG. 4(b), light emitting diode 22 produces light which is focused using a microlens set 24. The light strikes the cells and the resulting light is focused through filter 25 onto detector 26. The focused light beam can have power as low as 1 μW, with the required excitation density being as low as 1 W/m². The output of the detector is processed to determine the type of cell. One manner of detecting the cells is the use of a fluorescent emission. Thus, certain cells can be identified by their distinct fluorescent responses. Thus, certain cells may produce a strong green fluorescence when passing through the focused beam while the other cells produce different results. The fluorescence can be as low as 1 nW. However, other types of optical sorting may be used based on differences and other optical parameters. This may include scattering, the Raman spectrum, the cell size, the cell shape, the refractive index and so on. Accordingly, it is possible to separate different types of material using different types of optical parameters.

The microlens set can involve any arrangement of lenses which focus the light to an appropriate place and volume. In the arrangement of FIG. 4(c), two microlens sets are employed. The first is in front of the light emitting diode to focus the light to a volume about the size of the cell. The other is used for collecting and collimating the emission of fluorescence to the photo detector. In some cases, a band pass optical filter may be employed to only allow light in a specific range of wavelength to pass. Thus, the cells can be discriminated one at a time by monitoring the emission power and spectrum using a photodetector or a spectrometer. As seen in FIGS. 4(a) and 4(c), the optical detection arrangement is integrated with the microchannel structure. Thus, the integrated optical subsystem includes an embedded light source; the micro lens array and the photo detector along with associated other optical elements. The photodetector is commonly a bare chip of photodiode or avalanche photodiode (APD) made by silicon, SiC or InGaAs etc for measuring the optical power (18). The light source can be a light emitting diode, a laser diode or an organic light emitting diode.

### Cell Switching Using Flow Steering

Referring to FIG. 1, the cells are switched into different branches leading to different output wells by the appropriate control of switching wells 34. When a first type of cell is detected according to the optical parameters, a controller determines the amount of time it will take for the cell to reach the switching region and at that point activates the corresponding switching branch. Thus, if the cell is to go to the outlet well on the right, the left switching branch will be activated and push the cell to the right branch. This is known as “injection switching”. The electroosmotic force is used to steer the flows. In this way, fast acting and automatic cell sorting can be achieved.

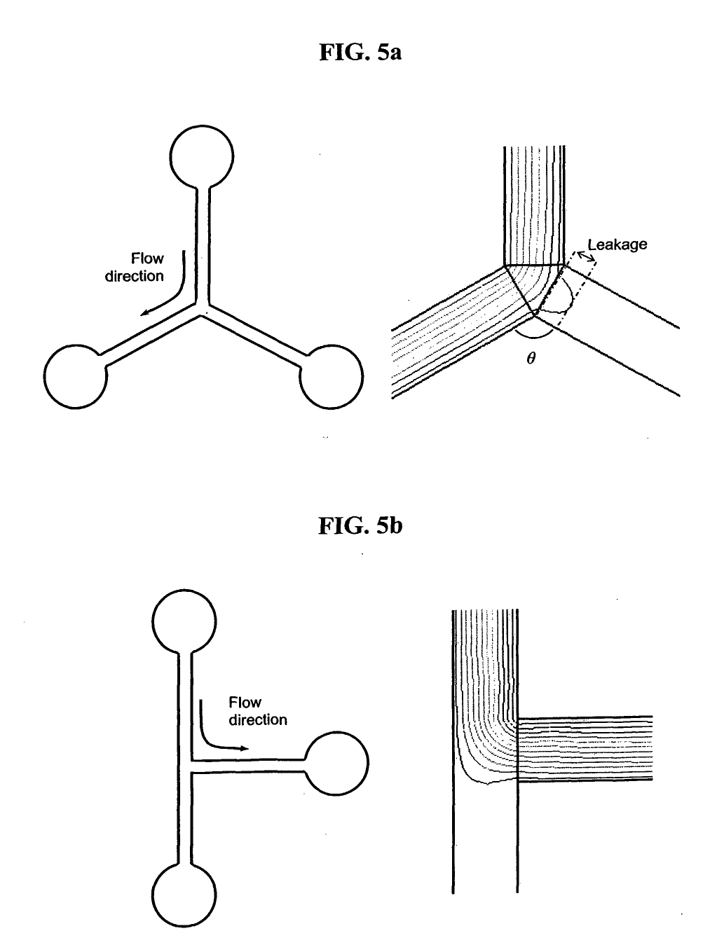

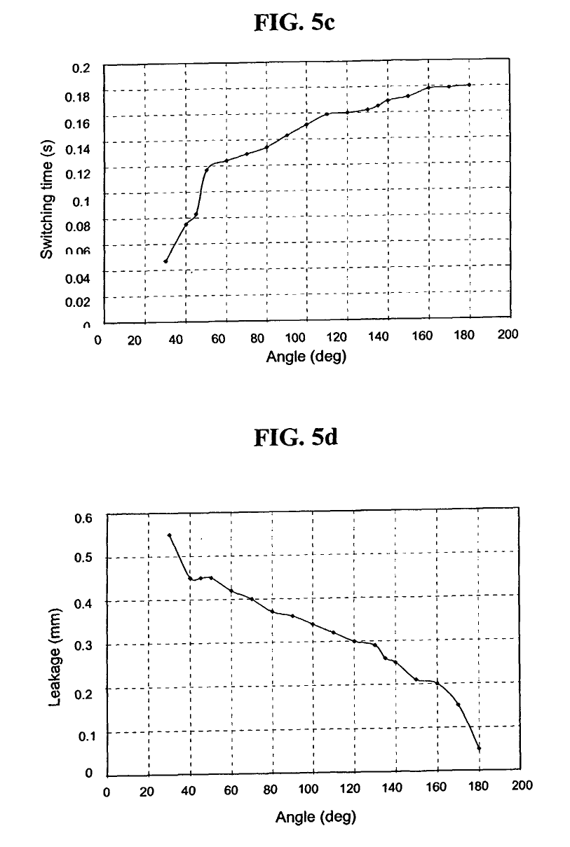

FIGS. 5(a) to 5(d) describe the switching time and flow leakage which are involved in the cell sorting. The graphs shown in FIGS. 5(c) and 5(d) describe the switching time required and the leakage amount involved for various angles between the incoming flow direction and the outgoing flow direction. The curves indicate that the optimal angle between the two channel branches are forty-five degrees, at which point the switching time is kept small while the leakage is also small. FIG. 5(a) shows a diagram of a forty-five degree angle and the resulting leakage which occurs is indicated. FIG. 5(b) indicates a 90° angle and also indicates the appropriate leakage. A controller (not shown) receives the output of the optical signal processing device 28 and controls the application of electrical signals for the electrodes surrounding the various wells. The controller is connected to the electrodes by way of wires made of gold, platinum or other metals or conductive polymers. Sorted cells can be retrieved by various meanings, for example, using a micropipette to suck them out.

### Example of Fabrication of Cell Sorter System

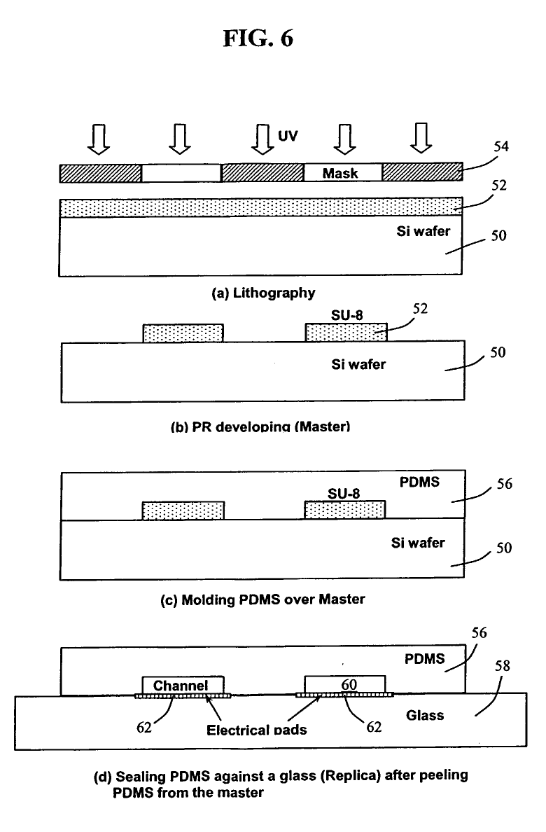

FIG. 6 shows a series of steps in the fabrication of the cell sorter system. In this example, the system is fabricated by soft lithography using PDMS and sealed with a glass slide. A master is first produced using photolithography technology. The design is transferred onto a photomask 54 with a high resolution down to 1 um. This photomask is used in contact lithography to produce a master with a negative-tone UV photoresist SU-8, 52, on a silicon wafer, 50. PDMS 56 is then poured over the master for molding.

The PDMS includes two components, a base and a curing agent. They are thoroughly mixed in an appropriate weight ratio (e.g. 10:1). After being poured into the master, the mixture is left for a time, such as a half an hour, so that air bubbles are released. Then the mixture is thermally cured (e.g. at 60-70° C. for 1 hour). After that, the PDMS replica is peeled off from the master. If desired, inlets, outlets and wells can be punched using circular metal punch pliers or a similar apparatus.

The microfluidic structure is thus formed with the bottom side open. A glass slide is then bonded to the molded microfluidic structure. It is possible to pattern electrodes 62 and wires on the glass slide 58 before bonding using thin film deposition and photolithographic etching (17). Channels 16 are formed in the PDMS due to the pattern of the photoresist in the master. These channels can appropriately be placed over the electrical pads as needed. It is also possible to embed the optical detection subsystem into the microfluidic system during molding by putting the optical subsystem onto the mold before pouring the soft material for molding and baking. Thus, the light sources, optical fibers and other optical structures may be thus embedded. The micro lens sets may be patterned and molded along with the microfluidic structures. Due to the photolithography, the micro lenses may have only vertical sidewalls rather than a spherical shape. The peeling-off and bonding may induce some distortion. However, small distortion does not significantly affect the functions. The purpose of using microlens sets is to avoid rapid divergence of light source and to collect more illumination light to the particles and the scattering/fluorescence light to the photodetector/spectrometer.

Since PDMS is elastometric, it can be sealed to a smooth surface without distorting the channels since no force or deformation is needed in the plasma bonding process. A reversible seal formed by simple van der Waals contact is watertight but can not withstand pressures greater than about ˜5 psi. In order to obtain an irreversible seal, the PDMS and the smooth surface of the hard substrate are exposed to oxygen plasma for a time (such as 1 minute, and are then bonded together. PDMS that has been molded against a smooth surface can conformally contact other smooth surfaces, even if they are nonplanar. For the plasma bonding, the two substrates are placed in a RIE machine (Technics series 800-IIC) and oxidized for 1 minute (19). The oxygen plasma is generated from oxygen gas at 75-mTorr throttle pressure, 75-sccm gas flow rate, and 100-W RF power. The oxygen plasma is formed by seeding the oxygen gas with a spark from a Tesla unit, the ions in the plasma reacting chemically with the surface of PDMS by oxidation of methyl groups to generate silanol groups (Si OH). Within 30 seconds after removal from the RIE machine, the substrates are brought into conformal contact and an irreversible seal is formed spontaneously. To maintain a strong hydrophilicity of the surface, the microfluidic system should be immediately filled with buffer solutions (such as 10 mMPBS buffer solution (138 mM NaCl, 2.7 mM KCl)) at pH 7.4.

### Clinical Use Example

### CD4/CD8 Ratio

This invention has many clinical applications. The ratio of CD4-type T cells to CD8-type T cells in a patient's blood is an important clinical parameter in management of HIV. Here as an example, the cell sorting system is used to measure the ratio of CD8 to CD4 cells in a sample. However, it should be noted that this example is to help explain more clearly the function and usefulness of this invention, it does not imply that the invention is only limited to this test.

T cells are special types of cells that are critical in the maintenance of the body's immune system. The AIDS virus attacks the immune system, and the absence of certain types of T cells plays a prominent role in being able to determine the progression of the HIV infection. The ratio of two specific types of T cells, known as CD4 and CD8 cells, can be used to monitor the progression of HIV infection to AIDS. During the course of an infection, the number of CD8 cells remains constant, while the number of CD4 cells falls precipitously. Thus, the ratio of CD4/CD8 T cells is an important indicator of HIV infection and development within the patient's body. The ratio in immune-competent adults is 2:1, or twice as many CD4 cells as CD8 cells. But during the course of HIV disease this ratio inverts, as CD8 cells expands while CD4 cells drop. As an example, in an uninfected adult, the CD4/CD8 ratio would be, for example, 1000 per deciliter/500 per deciliter (2.0), but with HIV this reverses, for example 450 CD4 per deciliter/900 CD8 per deciliter which equals 0.5. Decreases in this ratio for persons with HIV disease in the early stage and also a drop in the number of CD4 cells, for example to only 150 per deciliter, are signs of disease progression.

The cell sorter contains microfluidic circuitry for whole-blood sample acquisition, fluorescent-labeling of CD4 cells, continuous lysing of red blood cells, electrokinetic focus of leukocytes into a cell-sized narrow stream, and counting and sorting of CD4 and CD8 cells.

The inlet well 12 is preloaded with reagents such as ethylene diamine tetra acetic acid (EDTA) anticoagulants, CD4+ and CD8+ antibody tagged with fluorescent dye and saline solution (20). A cover should be put on the wells to avoid evaporation in storage. In clinical practices, the cover should be first removed, then a drop of blood is dropped into the inlet well. The focus well 14 is filled with the red blood cells (RBC) lysis buffer (e.g., eBioscience 1×RBC lysis buffer, Cat. No. 00-4333). At the inlet well 12, the CD4 and CD8 cells are bound to fluorescent labeled CD4+ and CD8+ antibodies. Sample solution is transported by the EOF, which is controlled by the platinum electrodes at the wells. At the intersection between the main and focus channels, continuous lysing of RBC and electrokinetic focus of the cell suspension are initiated. All the RBCs are lysed and the CD4 and CD8 cells are labeled with green and red fluorescent dye, respectively. Unbound antibodies are diluted by diffusing into adjacent sheath flows, resulting in adequately low level of background noise. The focusing effect enables a single cell suspension along the center line of the micro-channel and through the detection region, which permits more sensitive measurements to be made. The pre-focused sample moves down to the detection region where fluorescence is excited by the focused laser light then measured by an on-chip photodetector. CD4 and CD8 cells are identified by their distinct fluorescent responses. Peaks of fluorescent signal are counted using a data acquisition (DAQ) card (21), which corresponds to the number of CD4 and CD8 cells. The sorting process is realized by biasing the direction of the electroosmotic flow through electrically switching the voltages at output reservoirs.

The measurement of the CD4/CD8 ratio can be well implemented using this inventive cell sorting system.

### INDUSTRIAL APPLICABILITY

The subject cell sorter system is usable in a laboratory setting for a medical diagnosis and biological studies.

### REFERENCES CITED

The following references are hereby incorporated by reference in their entirety and for all purposes.
1. N. Catsimpoolas, Ed., Methods of cell separation. New York: Plenum, 1997.
2. - -, “Separation and purification: Critical needs and opportunities,” National Academy of Sciences-Nat. Res. Council, Washington, D.C., Report of the Committee of Separation Science and Technology, 1987.
3. D. J. Harrison, K. Fluri, K. Seiler, Z. H. Fan, C. S. Effenhauser, and A. Manz, “Micromachining a miniaturized capillary electrophoresis-based chemical analysis system on a chip,” Science, vol. 261, pp. 859-897, 1993.
4. A. Manz, N. Graber, and H. M. Widmer, “Miniaturized total chemical analysis systems: a novel concept for chemical sensing,” Sensors and Actuators B, vol. 1, no. 3, pp. 244-248, 1990.
5. N. H. Chiem and D. J. Harrison, “Monoclonal antibody binding affinity determined by microchip-based capillary electrophoresis,” Electrophoresis, vol. 19, pp. 3040-3044, 1998.
6. J. F. Li, J. J. Kelly, I. Chemushevich, D. J. Harrison, and P. Thibault, “Separation and identification of peptides from Gel-isolated membrane proteins using a microfabricated device for combined capillary electrophoresis/nanoelectrospray mass spectrometry,” Analytical Chemistry, vol. 72, pp. 599-609, 2000.
7. P. C. H. Li and D. J. Harrison, “Transport, manipulation, and reaction of biological cells on-chip using electrokinetic effects,” Analytical Chemistry, vol. 69, pp. 1564-1568, 1997.
8. J. Kruger, K. Singh, C. Jackson, A. Morrison, “Development of a microfluidic device for fluorescence activated cell sorter,” Journal of Micromechanical and Microengineering vol. 12, no. 4, pp. 486-494, 2002.
9. A. Y. Fu, C. Spence, A. Scherer, S. R. Quake, “A microfabricated fluorescence-activated cell sorter,” Nature Biotechnology, vol. 17, no. 11, pp. 1109-1111, 1999.
10. P. S. Dittrich, P. Schwille, “An integrated microfluidic system for reaction, high-sensitivity detection, and sorting of fluorescent cells and particles,” Analytical Chemistry, vol. 75, no. 21, pp. 5767-5774, 2003.
11. J. Wang, “On-chip enzymatic assays,” Electrophoresis, Vol. 23, no. 5, pp. 713-718, 2002.
12. A. Hatch, A. E. Kamhols, K. R. Hawkins, M. S. Munson, “A rapid diffusion immunoassay in a T-sensor,” Nature Biotechnology, vol. 19, no. 5, pp. 461-465, 2001.
13. M. U. Kopp, A. J. de Mello, A. Manz, “Chemical amplification: continuous-flow PCR on a chip,” Science, vol. 280, no. 5366, pp. 1046-1048, 1998.
14. C. F. Spense, An. Y. Fu, S. R. Quake, and F. H. Arnold, “Microfabricated cell sorter for chemical and biological materials,” U.S. Pat. No. 6,540,895 (2003).
15. J. C. Mcdonald and G. M. Whitesides, “Poly(dimethylsiloxane) as a material for fabricating microfluidic devices,” Acct. Chem. Res., vol. 35, no. 7, pp. 491-499, 2002.
16. L-M Fu, R-J Yang, C-H Lin, Y-J Pan, G-B Lee, “Electrokinetically driven micro flow cytometers with integrated fiber optics for on-line cell/particle detection,” Analytica Chimica Acta, vol. 507, pp. 163-169, 2004.
17. - , “An Introduction to MEMS (Micro-electromechanical Systems)”, Prime Faraday Partnership, January 2002.
18. More information in http://www.lasercomponents.de/.
19. B. H. Jo, L. M. VanLerberghe, K. M. Motsegood and D. J. Beebe, “Three-dimensional micro-channel fabrication in polydimethylsiloxane (PDMS) elastomer,” Journal of Microelectromechanical System, vol 9, pp. 76-81, 2000.
20. Z. Darzynkiewicz, H. A. Crissman and J. P. Robinson, Ed, Cytometry. Part B. San Diego, Calif.: Academic Press, 2001.
21. The DAQ cards from National Instruments are widely used. See http://www.ni.com/dataacquisition/.

What is claimed is:

1. A cell sorter system for sorting cells in a solution,
which system comprises:
a substrate;
a layer including a microfluidic structure mounted onto said substrate, said microfluidic structure forming microchannels;
an optical subsystem which is integrated with said layer;
a cell discrimination system for receiving a signal from said optical subsystem and recognizing cells thereby;
said discrimination system converting the received signal from said optical subsystem into an electrical signal for retrieving information of cells; said discrimination system being at least partially integrated onto the substrate, or being external to the substrate and having optical coupling with the optical subsystem; and
an electrical circuit for controlling flows between a least two microchannels based upon a signal from said cell discrimination system.

2. The cell sorter system of claim 1, wherein the substrate is a hard surface.

3. The cell sorter system of claim 1, wherein the hard surface is one of a glass slide, silicone wafer or a polymer slab.

4. The cell sorter system of claim 1, wherein the optical detection subsystem further comprises:
a light source;
at least one micro lens set for focusing light from said
light source to a desired beam size within a channel; and a
a photo detector for receiving light from said cells.

5. The cell sorter system of claim 4, wherein the light source is an LED light source and is embedded into said layer.

6. The cell sorter system of claim 4, wherein the light source is an LED light source and is embedded into said layer, and wherein said photodetector is embedded into the layer.

7. The cell sorter system of claim 4, wherein the light source is an LED light source and is embedded into said layer, wherein said at least one micro lens set includes a first micro lens set embedded into the layer focusing light from the source onto said cells or particles to be sorted and a second micro lens set embedded in the layer for collecting light from the cells or particles for transmission to a photodetector.

8. The cell sorter system of claim 1, wherein the electrical circuit applies an electrical field to steer the solution carrying cells.

9. The cell sorter system of claim 8, wherein the electrical circuit includes a controller connected to said optical subsystem for receiving signals recognizing cells; and a plurality of electrodes connected to said controller by wires so that said controller steers said cells in solution to sort cells; said controller being hybridly integrated onto the substrate or a separate component external to the substrate; and said controller being a data processing center to recognize said cells through the electrical signal from the discrimination system and to adjust a level and direction of a potential applied to electrical pads for steering the flow direction.

10. The cell sorter system of claim 1, wherein said microfluidic structure contains and transports said cells in solution, said microfluidic structure including:
a plurality of wells for inlets and outlets;
a plurality of focusing channels for cell focusing by a flow control;
a main channel for carrying said cells in solution; and
a plurality of branch channels connected to output wells for carrying separated cells.

11. The cell sorter system of claim 10, wherein said focusing channels intersect in a focusing region and are connected to the same well so as to have the same hydrostatic pressure in said focusing channels.

12. The cell sorter system of claim 10, wherein an angle between said branch channels is no less than 45°.

13. The cell sorter system of claim 1, wherein the received signal is one of projection, scattering, fluorescence, interference and diffraction.

14. The cell sorter system of claim 1, wherein the information of cells is one of cell size, shape and optical refractive index.

15. A method of optical detection of particles in a solution comprising:
providing a micro lens set to focus input light from a light source to a beam having a size approximately equal to said particle; said micro lens set having a size of several micrometers to several millimeters, and being cylindrical shape fabricated photolithographically on the substrate, or traditional spherical lenses fabricated separately and later integrated onto the substrate;
shining said focused light beam onto said particles, whereby said focused light beam illuminates one particle at a time and excites said particles to fluoresce; and
said focused light beam having low power down to 1 μW and the required excitation density being as low as 1 W/m² to as high as 10⁹ W/m²; and said fluorescence being as low as 1 nW to as high as 10 mW.

16. A method of optical detection according to claim 15, further comprising: providing a micro lens set to collect output light from said cells.

17. A method of cell or particle sorting comprising the steps of:
providing a plurality of wells connected to an input by branch channels;
applying a high potential between the input and a desired destination well while biasing other wells at a lower potential so as to switch cells in a solution to the desired destination wells due to an electroosmotic force causing switching of flow direction.

18. A method of fabricating a cell sorter system comprising the steps of:
forming a master for molding;
applying a polymeric material onto said master and curing the polymeric material whereby a microfluidic structure is formed in the polymeric material;
removing said the cured material from said master;
applying said microfluidic structure to a hard substrate to form said cell sorter system.

19. The method of fabrication of a cell sorter system according to claim 18, further comprising forming electrodes and wires on a surface of said hard substrate aligned with microfluidic structure for electrical field control; said electrodes and wires being on the same side as the microchannel structures for flow steering, and extended to the opposite side for easy connection to external components.

20. The method of fabrication of the cell sorter system according to claim 18, further comprising forming electrodes and wires on the microfluidic structure for electrical field control; said electrodes and wires being first fabricated by photolithographically patterning a thin layer of deposited metal or conductive polymer on the hard substrate, then the removed microfluidic structure is bonded on top of the hard substrate with the electrodes aligned to wells, inlets and outlets of the microfluidic structure.

21. The method of fabrication of a cell sorter system according to claims 18, further comprising a step of punching inlet and outlet wells in said microfluidic structure using a punching tool, or by fabricating the pins on the mold.

22. The method of fabrication according to claim 21, wherein said punching tool is metal punch pliers or an automatic punch machine.

23. The method of fabrication according to claim 18, further comprising a step of exposing the joined microfluidic structure and the hard substrate to a plasma for 3 seconds to ten minutes.

24. The method of fabrication of the cell sorter system according to claim 23, further comprising the step of filling the microfluidic structure with a buffer solution after plasma bonding to maintain a strong hydrophilic property of the microchannel surface.

25. The cell sorter system of claim 1, wherein said microfluidic structure includes an angled microchannel structure between collection wells.

26. The cell sorter system of claim 1, wherein the angle is 45°.

27. A cell sorter system for sorting cells in a solution, comprising a plurality of analysis units, each analysis unit including:
a substrate;
a microfluidic structure mounted onto said substrate to form channels;
an optical subsystem for recognizing cells which is integrated with said microfluidic structure;
an electrical circuit for sorting cells based on said cell recognition;
wherein said analysis units are cascaded so that an output of a first analysis unit serves as an input to an second analysis unit.

28. Use of the cell sorter system of claim 1 to measure a ratio of CD4 to CD8 T cells in a sample.

29. Use of the cell sorter system of claim 1 to separate and count cells according to a desired property.
